(19) United States
(12) Patent Application Publication
(10) Pub. No.: US 2024/0145061 A1
(43) Pub. Date: May 2, 2024

(54) AUTOMATED HOME INFUSION PHARMACIST EXPERIENCE

(71) Applicant: Brightree LLC, Peachtree Corners, GA (US)

(72) Inventors: Christopher Wesley LIEU, Peachtree Corners, GA (US); Nupura KOLWALKAR, Cumming, GA (US); Kayla Grunder BYINGTON, Urbandale, IA (US)

(21) Appl. No.: 18/494,480

(22) Filed: Oct. 25, 2023

Related U.S. Application Data
(60) Provisional application No. 63/420,431, filed on Oct. 28, 2022.

(51) Int. Cl. G16H 20/17

(52) U.S. Cl. CPC G16H 20/17

(57) ABSTRACT

Techniques for patient medical care are disclosed. These techniques include identifying a plurality of prescriptions for fulfillment by a pharmacist for a patient. The techniques further include providing the prescriptions for presentation on a first screen of a user interface (UI) and receiving a selection of at least two of the plurality of prescriptions, based on input to the UI. The techniques further include identifying supplies associated with the at least two prescriptions, and providing the supplies for presentation on a second screen of the UI that is accessible based on input to the first screen. The techniques further include receiving a selection of a first supply, based on input to the UI, and fulfilling the at least two prescriptions and the first supply, including triggering delivery of the at least two prescriptions and the first supply for treatment of the patient using a medical therapy.

### CROSS-REFERENCE TO RELATED APPLICATIONS

This application claims priority to U.S. Provisional Patent Application No. 63/420,431, filed Oct. 28, 2022, the entire content of which is incorporated herein by reference in its entirety.

### INTRODUCTION

Aspects of the present disclosure relate to patient medical care, and more specifically, to improved medical item fulfillment.

Providing medical items to patients is very challenging. This is particularly true for home infusion therapies, which typically involve the intravenous or subcutaneous administration of drugs or biologicals to an individual at home. Many different components are needed to perform home infusion, including the prescription medication, equipment (e.g., a pump or other delivery or monitoring device), and other supplies (e.g., tubing, catheters, and other associated supplies). The home infusion process typically requires coordination among multiple entities, including patients, physicians, hospital discharge planners, health plans, home infusion pharmacies, and, if applicable, home health agencies, among other entities. Further, the home infusion process is typically fulfilled and delivered manually, requiring a pharmacist or other medical professional to manually select items, one-by-one, for delivery to patients. This is time consuming for pharmacists, and a significant detriment to patient outcomes, because of delays, errors in providing necessary items (e.g., forgotten, missing, or incorrect items), and other drawbacks.

### SUMMARY

Embodiments include a computer-implemented method. The method includes identifying a plurality of prescriptions for fulfillment by a pharmacist for a patient. The method further includes providing the plurality of prescriptions for presentation on a first screen of a user interface (UI). The method further includes receiving a selection of at least two of the plurality of prescriptions, based on input to the UI. The method further includes identifying one or more supplies associated with one or more of the at least two prescriptions, and providing the supplies for presentation on a second screen of the UI, wherein the second screen of the UI is accessible based on input to the first screen. The method further includes receiving a selection of a first supply of the one or more supplies, based on input to the UI. The method further includes fulfilling the at least two prescriptions and the first supply, including: triggering delivery of the at least two prescriptions and the first supply for treatment of the patient using a medical therapy.

Embodiments further include an apparatus including a memory and a hardware processor communicatively coupled to the memory, the hardware processor configured to perform operations. The operations include identifying a plurality of prescriptions for fulfillment by a pharmacist for a patient. The operations further include providing the plurality of prescriptions for presentation on a first screen of a user interface (UI). The operations further include receiving a selection of at least two of the plurality of prescriptions, based on input to the UI. The operations further include identifying one or more supplies associated with one or more of the at least two prescriptions, and providing the supplies for presentation on a second screen of the UI, wherein the second screen of the UI is accessible based on input to the first screen. The operations further include receiving a selection of a first supply of the one or more supplies, based on input to the UI. The operations further include fulfilling the at least two prescriptions and the first supply, including: triggering delivery of the at least two prescriptions and the first supply for treatment of the patient using a medical therapy.

Embodiments further include a non-transitory computer-readable medium including instructions that, when executed by a processor, cause the processor to perform operations. The operations include identifying a plurality of prescriptions for fulfillment by a pharmacist for a patient. The operations further include providing the plurality of prescriptions for presentation on a first screen of a user interface (UI). The operations further include receiving a selection of at least two of the plurality of prescriptions, based on input to the UI. The operations further include identifying one or more supplies associated with one or more of the at least two prescriptions, and providing the supplies for presentation on a second screen of the UI, wherein the second screen of the UI is accessible based on input to the first screen. The operations further include receiving a selection of a first supply of the one or more supplies, based on input to the UI. The operations further include fulfilling the at least two prescriptions and the first supply, including: triggering delivery of the at least two prescriptions and the first supply for treatment of the patient using a medical therapy.

The following description and the related drawings set forth in detail certain illustrative features of one or more embodiments.

### DESCRIPTION OF THE DRAWINGS

The appended figures depict certain aspects of the one or more embodiments and are therefore not to be considered limiting of the scope of this disclosure.

FIG. 1 depicts a computing environment for automated home infusion fulfillment, according to one embodiment.

FIG. 2 depicts a block diagram for a controller for automated home infusion fulfillment, according to one embodiment.

FIG. 3 is a flowchart illustrating automated home infusion fulfillment, according to one embodiment.

FIG. 4 is a flowchart illustrating adding supplies for automated home infusion fulfillment, according to one embodiment.

FIG. 5 is a flowchart illustrating generating orders for automated home infusion fulfillment, according to one embodiment.

FIG. 6 illustrates a selection interface for automated home infusion fulfillment, according to one embodiment.

FIG. 7 illustrates a prescription refill interface for automated home infusion fulfillment, according to one embodiment.

FIG. 8 illustrates a further prescription refill interface for automated home infusion fulfillment, according to one embodiment.

FIG. 9 illustrates another further prescription refill interface for automated home infusion fulfillment, according to one embodiment.

FIG. 10 illustrates a supply fulfillment interface for automated home infusion fulfillment, according to one embodiment.

FIG. 11 illustrates a review interface for automated home infusion fulfillment, according to one embodiment.

FIG. 12 illustrates an order interface for automated home infusion fulfillment, according to one embodiment.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

Aspects of the present disclosure provide apparatuses, methods, processing systems, and computer-readable mediums for improved fulfillment of medical items. As discussed above, providing medical items, especially prescription items, is very challenging. This is particularly true for home infusion therapies. Existing systems require significant manual intervention and analysis, which results in risks to patient outcomes and many computational inefficiencies.

One or more techniques disclosed herein relate to automated home infusion fulfillment. In an embodiment, a medical professional (e.g., a pharmacist) can select multiple prescription items for fulfillment using a unified, end-to-end, automated interface. The medical professional can select which item(s), among several possible choices, should be fulfilled. Further, the medical professional can add additional items associated with the therapies. This can include equipment, additional supplies other than equipment, and any other suitable items. In an embodiment, a user interface (UI) is automatically populated with potentially relevant items for fulfillment.

In an embodiment, the automated system further generates a unified order for all products for fulfillment, and a delivery ticket (e.g., that triggers delivery of the products). This allows a medical professional to use a unified, automated, end-to-end interface to fulfill home infusion therapy items. While the embodiments discussed herein are focused on home infusion therapies, this is merely an example. One or more of these techniques can be used for fulfillment of any suitable medical item.

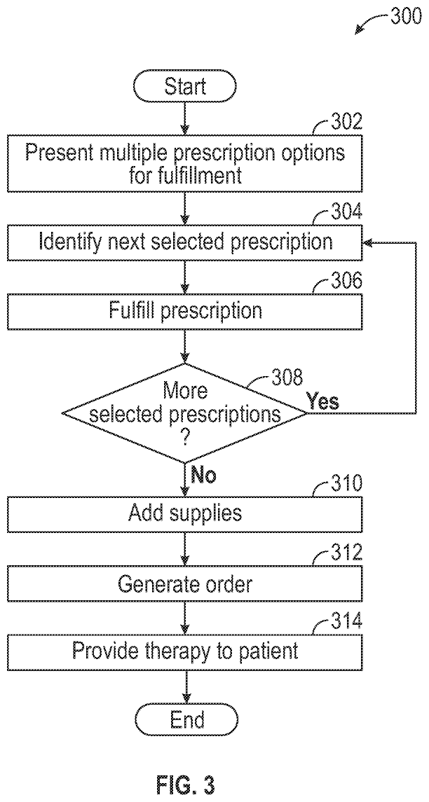

In an embodiment, one or more of these techniques provide technical improvements to existing medical supply systems. For example, existing user interfaces for pharmacists to prescribe home infusion therapy items are inefficient, both computationally and in user accessibility. Pharmacists typically manually prescribe each item, and manually select supplies to accompany prescriptions. One or more techniques discussed herein can be used to automate, streamline, and improve this process. For example, a unified UI workflow can be used to allow a pharmacist to fulfill multiple prescriptions, add supplies, and complete a sales order. This eliminates redundant computational operations needed to fulfill individual prescriptions. Network transmissions with electronic databases (e.g., for security authentication and prescription fulfillment) can be reduced by replacing one-by-one fulfillment of prescriptions with a streamlined process described below in relation to FIGS. 3-12. And computational resources can be saved by replacing repeated authentication operations and repeated overhead associated with existing techniques with a streamlined process described below in relation to FIGS. 3-12.

Further, in an embodiment, one or more techniques described herein provide for improved medical treatment for patients. As discussed, in existing solutions for home infusion therapy fulfillment pharmacists typically manually prescribe each item, and manually select supplies to accompany prescriptions. This can lead to significant errors, and increased time for patients to receive needed therapy, and can even lead patients to discontinue or avoid medically valuable home infusion therapies. For example, in existing systems a pharmacist might miss prescriptions or supplies that would improve patient outcomes. As described below in relation to FIGS. 3-12, this can be improved by a unified UI workflow that presents to a pharmacist a list of relevant prescriptions (e.g., all relevant prescriptions for a given therapy or combination of therapies), and a list of associated supplies (e.g., all suitable supplies or a subset of recommended supplies). Further, these improvements can reduce mistakes by pharmacists (e.g., incorrect prescriptions or supply orders) with a unified UI. And these improvements can save significant time and frustration for pharmacists, reducing wait times for patients and making it easier for patients to receive therapies, and less likely for patients to discontinue therapies recommended by care providers.

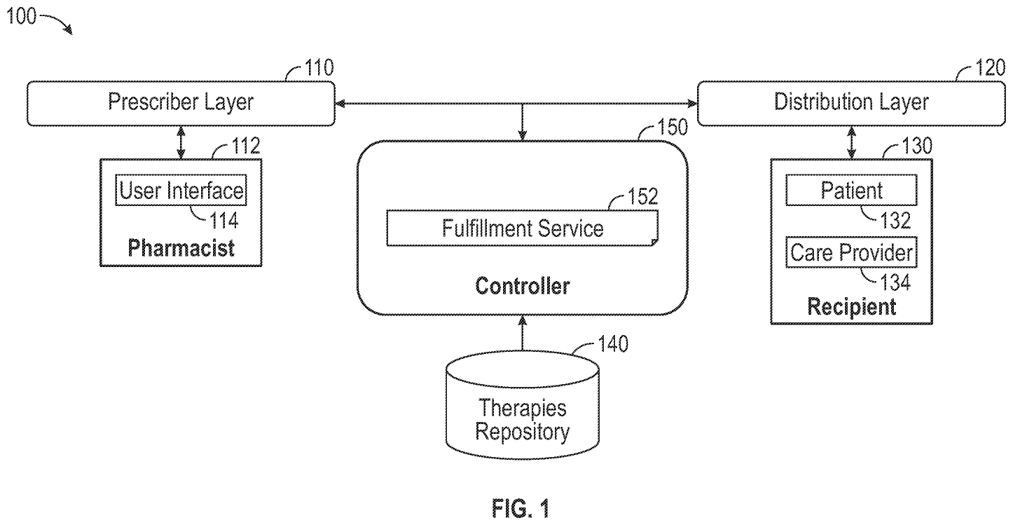

FIG. 1 depicts a computing environment 100 for automated home infusion fulfillment, according to one embodiment. In an embodiment, a pharmacist 112 (or another suitable medical professional) can interact with a prescriber layer 110 using a user interface 114. In an embodiment, the pharmacist 112 can use any suitable electronic or computing device, including a smartphone, a tablet, a laptop computer, a desktop computer, a wearable device, a medical device, an Internet of Things (IoT) device, or any other suitable device. For example, the pharmacist 112 can use a device that includes a user interface 114 to present information to the pharmacist 112. The user interface 114 can present information to the pharmacist 112 visually (e.g., using a display screen), through audio (e.g., using one or more speakers), or using any other suitable technique. Further, the user interface 114 can allow the pharmacist 112 to interact with the electronic device (e.g., using a keyboard, mouse, touch sensitive interface, voice interface using one or more microphones, vision-tracking interface, or any other suitable interface).

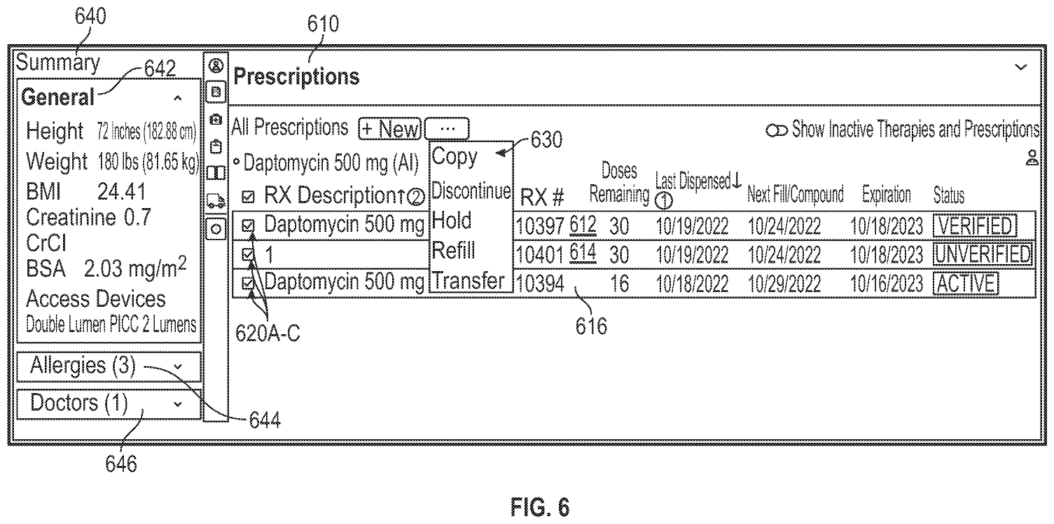

For example, the user interface 114 can include the interfaces illustrated in FIGS. 6-12, below. As discussed further below with regard to FIGS. 3-5, the pharmacist 112 can use the user interface 114 to select prescriptions for fulfillment, supplies for fulfillment, and to generate a sales order to complete the fulfillment.

In an embodiment, the user interface 114 interacts with a controller 150. In an embodiment, the controller 150 includes a fulfillment service 152. The fulfillment service 152 facilitates fulfilling prescriptions, and other medical item orders (e.g., supplies for home infusion), selected by the pharmacist 112 using the user interface 114. In an embodiment, the fulfillment service 152 further facilitates generating the user interface 114 (e.g., a web interface, mobile app interface, or other suitable interface). Alternatively, or in addition, another suitable service facilitates generating the user interface 114 (e.g., a UI service).

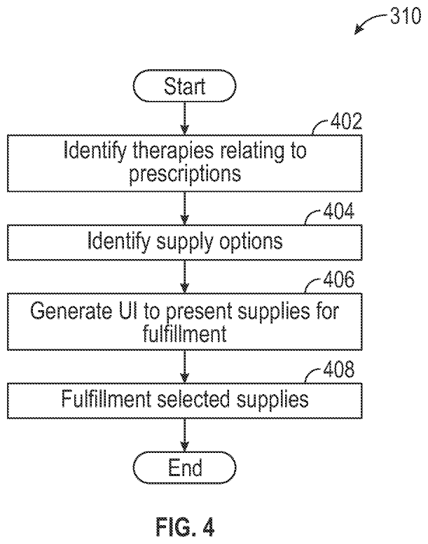

In an embodiment, the controller 150 uses a therapies repository 140 to identify information about therapies (e.g., home infusion therapies). For example, as discussed further below with regard to FIGS. 3-4, the fulfillment service 152 can identify supplies associated with a given therapy (or a given prescription) using the therapies repository 140. In an embodiment, the therapies repository 140 is any suitable electronic storage repository, including an electronic database (e.g., a relational database, a graph database, or any other suitable database), a cloud storage location (e.g., a public cloud, a private cloud, or a hybrid cloud), an on-site or remote storage location, a distributed storage location (e.g., a blockchain), or any other suitable electronic storage repository.

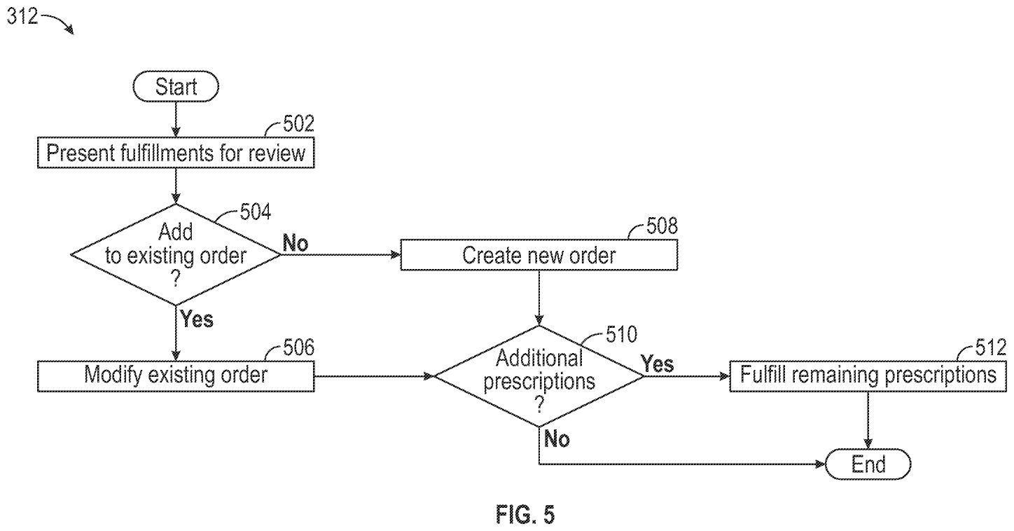

The controller 150 further interacts with a distribution layer 120 to provide the prescriptions, supplies, or both, to a recipient 130. For example, the fulfillment service 152 can generate or modify a sales order (e.g., as discussed below in relation to FIG. 5) which can be used to fulfill the selected items. In an embodiment the items are delivered to the patient 132 at home. For example, the items can be delivered to the patient 132 at home, and then a care provider (e.g., a nurse) can come to the patient's home and assist with administering the therapy, or the patient can administer the therapy directly. Alternatively, or in addition, the items can be delivered to a pharmacist or a care provider 134 working with the patient (e.g., a nurse or doctor), and the pharmacist or care provider 134 can assist with administering the therapy (e.g., at the patient's home, at a pharmacy, at a medical facility, or at any other suitable location).

In an embodiment, the various components of the computing environment 100 communicate using one or more suitable communication networks, including the Internet, a wide area network, a local area network, or a cellular network, and uses any suitable wired or wireless communication technique (e.g., WiFi or cellular communication). Further, in an embodiment, the prescriber layer 110, controller 150, distribution layer 120, and therapies repository 140, can be implemented using any suitable combination of physical compute systems, cloud compute nodes and storage locations, or any other suitable implementation. For example, the prescriber layer 110, controller 150, distribution layer 120, and therapies repository 140, could each be implemented using a respective server or cluster of servers. As another example, the prescriber layer 110, controller 150, distribution layer 120, and therapies repository 140, can be implemented using a combination of compute nodes and storage locations in a suitable cloud environment. For example, one or more of the components of the prescriber layer 110, controller 150, distribution layer 120, and therapies repository 140 can be implemented using a public cloud, a private cloud, a hybrid cloud, or any other suitable implementation.

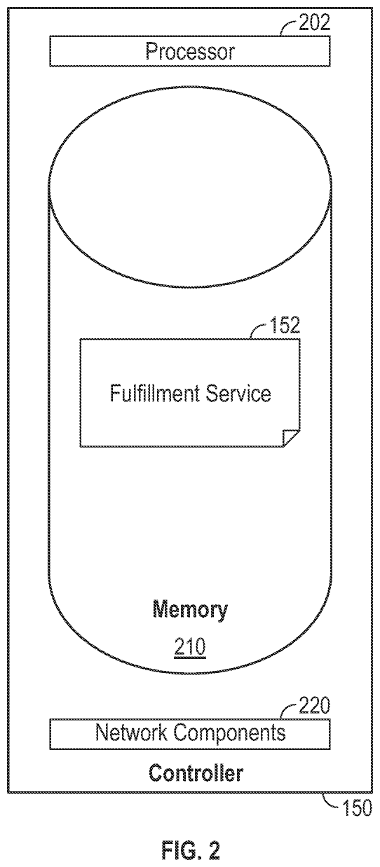

FIG. 2 depicts a block diagram for a controller 150 for automated home infusion fulfillment, according to one embodiment. The controller 150 includes a processor 202, a memory 210, and network components 220. The memory 210 may take the form of any non-transitory computer-readable medium. The processor 202 generally retrieves and executes programming instructions stored in the memory 210. The processor 202 is representative of a single central processing unit (CPU), multiple CPUs, a single CPU having multiple processing cores, graphics processing units (GPUs) having multiple execution paths, and the like.

The network components 220 include the components necessary for the controller 150 to interface with a suitable communication network (e.g., a communication network interconnecting various components of the computing environment 100 illustrated in FIG. 1, or interconnecting the computing environment 100 with other computing systems). For example, the network components 220 can include wired, WiFi, or cellular network interface components and associated software. Although the memory 210 is shown as a single entity, the memory 210 may include one or more memory devices having blocks of memory as sociated with physical addresses, such as random access memory (RAM), read only memory (ROM), flash memory, or other types of volatile and/or non-volatile memory.

The memory 210 generally includes program code for performing various functions related to use of the controller 150. The program code is generally described as various functional “applications” or “modules” within the memory 210, although alternate implementations may have different functions and/or combinations of functions. Within the memory 210, the fulfillment service 152 facilitates automated fulfillment for home infusion therapy. This is discussed further, below, with regard to FIGS. 3-12.

While the controller 150 is illustrated as a single entity, in an embodiment, the various components can be implemented using any suitable combination of physical compute systems, cloud compute nodes and storage locations, or any other suitable implementation. For example, the controller 150 could be implemented using a server or cluster of servers. As another example, the controller 150 can be implemented using a combination of compute nodes and storage locations in a suitable cloud environment. For example, one or more of the components of the controller 150 can be implemented using a public cloud, a private cloud, a hybrid cloud, or any other suitable implementation.

Although FIG. 2 depicts the fulfillment service 152 as being located in the memory 210, that representation is also merely provided as an illustration for clarity. More generally, the controller 150 may include one or more computing platforms, such as computer servers for example, which may be co-located, or may form an interactively linked but distributed system, such as a cloud-based system, for instance. As a result, the processor 202, and the memory 210, may correspond to distributed processor and memory resources within the computing environment 100. Thus, it is to be understood that the fulfillment service 152 may be stored at any suitable location within the distributed memory resources of the computing environment 100.

FIG. 3 is a flowchart 300 illustrating automated home infusion fulfillment, according to one embodiment. At block 302, a fulfillment service (e.g., the fulfillment service 152 illustrated in FIGS. 1-2) presents multiple prescription options for fulfillment. In an embodiment, the fulfillment service identifies multiple prescription options available for fulfillment, and presents these options on a UI. A pharmacist, or another suitable medical professional, selects prescriptions to fulfill.

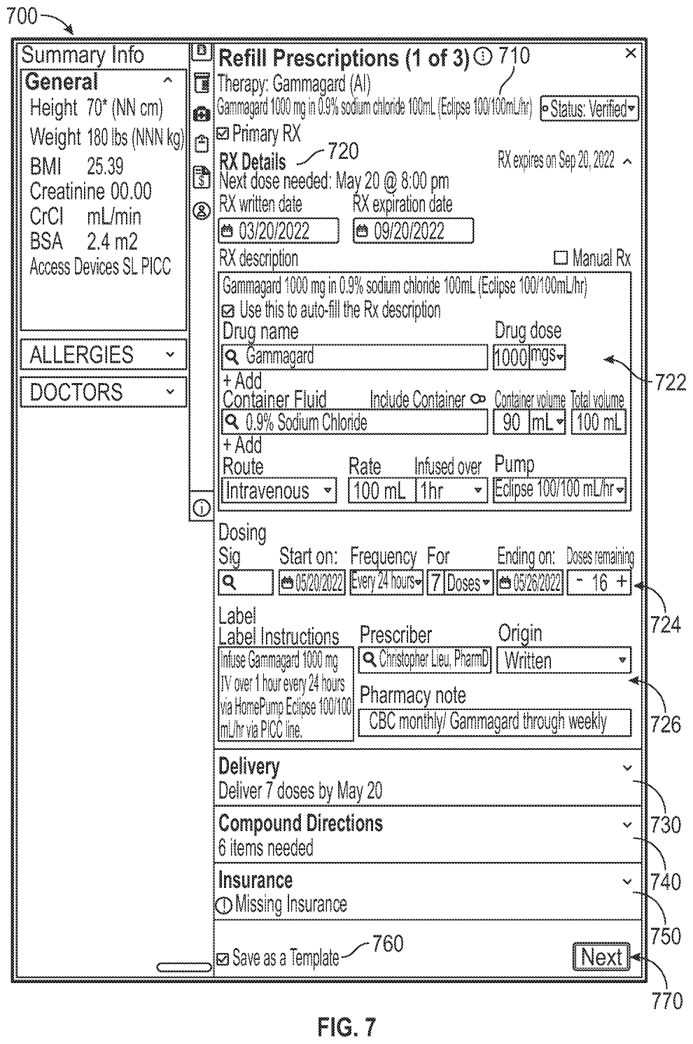

For example, as discussed above, a home infusion therapy often include multiple prescriptions to be fulfilled, together, to complete the therapy. The fulfillment service can identify these prescriptions based on an electronic repository (e.g., the therapies repository 140 illustrated in FIG. 1), or using any other suitable technique. FIGS. 6-9 illustrate an example UI for presenting these home infusion prescription options for fulfillment. In the example illustrated by FIG. 6, each of the prescriptions is presented on an initial UI screen with check-boxes allowing a pharmacist to select which prescriptions should be fulfilled. As illustrated in FIGS. 7-9, the pharmacist can then follow through additional UI screens, for each prescription, to complete fulfillment for each prescription. This is merely an example, and any suitable UI can be used for any suitable medical items.

In an embodiment, the fulfillment service generates the UI screens. Alternatively, another suitable service (e.g., a UI service) generates the UI screens. Further, the UI can be a graphical UI, an audio UI (e.g., in which a medical professional interacts with the UI using voice commands or other audio commands), a textual UI, or any other suitable UI.

At block 304, the fulfillment service identifies a next selected prescription. In an embodiment, a UI presents the multiple prescription options for selection by a pharmacist (e.g., as illustrated in FIG. 6). The pharmacist then selects one or more of the available options. The fulfillment service identifies the next selected option. Further, the fulfillment service (or another suitable service) generates a UI (e.g., as shown in FIGS. 7-9) to allow the pharmacist to fulfill the prescription.

At block 306, the fulfillment service fulfills the prescription. In an embodiment, the fulfillment service (or another suitable service) presents a UI to the pharmacist to allow the pharmacist to fulfill the prescription. For example, as illustrated in FIGS. 7-9, the pharmacist can select a variety of options for fulfilling the prescription (e.g., container, dosing, quantity, delivery, compounding directions, and many other suitable options). The fulfillment service can use these UI selections to generate an order, delivery ticket, or both, to fulfill the prescription. In an embodiment, the delivery ticket is used to trigger delivery of the fulfillment items. This can include direct, or indirect, delivery to any suitable location or entity, including a patient, a care provider associated with the patient, a medical facility associated with the patient, or any other suitable location or entity.

At block 308, the fulfillment service determines whether there are more selected prescriptions. In an embodiment, as discussed above in relation to block 302 and illustrated below in relation to FIG. 6, the fulfillment service can present to the pharmacist multiple prescription options (e.g., using check boxes or another suitable selection technique). The pharmacist can then select prescriptions for fulfillment, using the UI. In an embodiment, the fulfillment service iterates through these selections and presents fulfillment options to the pharmacist for each prescription, as illustrated in FIGS. 7-9.

If the fulfillment service determines that more selected prescriptions exist (e.g., not all selected prescriptions have been presented for fulfillment), the flow returns to block 304 and the fulfillment service presents the next prescription for fulfillment. Otherwise, the flow proceeds to block 310.

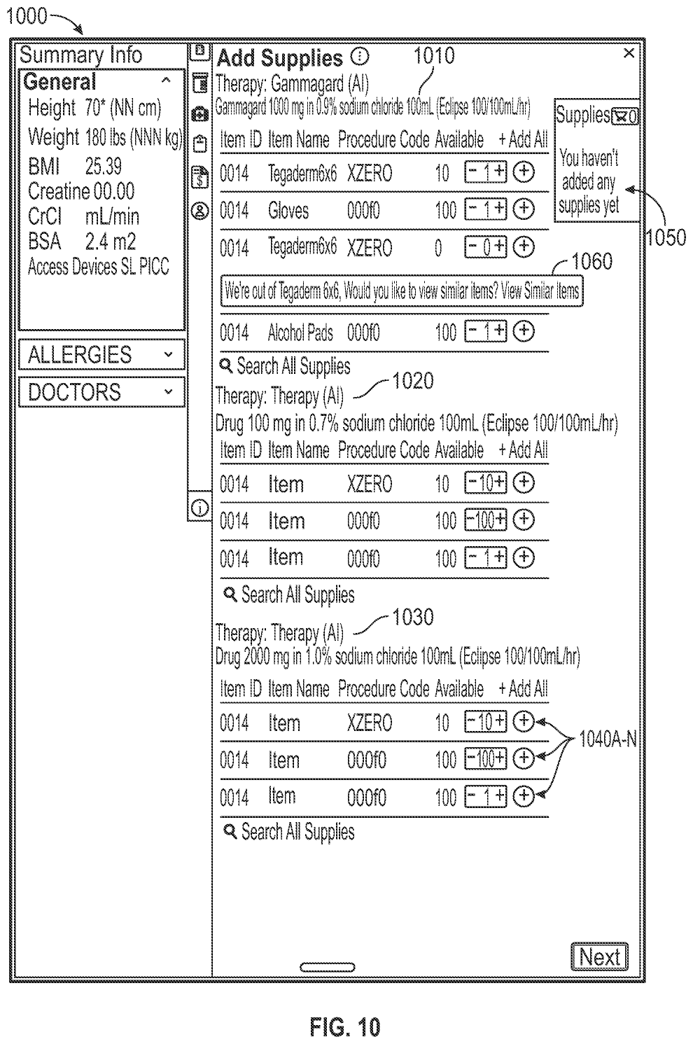

At block 310, the fulfillment service adds supplies. As discussed above, home infusion therapies typically involve both prescriptions and supplies (e.g., including equipment and other supplies). In an embodiment, the fulfillment service can identify supplies associated with the prescriptions selected for fulfillment. For example, the fulfillment service can use a repository of therapy information (e.g., the therapies repository 140 illustrated in FIG. 1) to identify supplies associated with each selected prescription. The fulfillment service can then present the supplies to the pharmacist for fulfillment, along with the prescription. The fulfillment service can present all supplies associated with the selected prescriptions, or a subset of supplies (e.g., recommended supplies). This is discussed further, below, with regard to FIGS. 4 and 10.

At block 312, the fulfillment service generates an order. For example, the fulfillment service can generate an order for fulfillment of the prescriptions, and supplies, selected by the user. In an embodiment, the fulfillment service can identify an existing order and modify the order by adding the additional items for fulfillment. Alternatively, the prescription service can create a new order. This is discussed further, below, with regard to FIGS. 5 and 11-12.

At block 314, the order is used to provide therapy to a patient. In an embodiment, the prescriptions and supplies are associated with a home infusion therapy. The order generated at block 312 can trigger delivery of the prescriptions and items to the patient (e.g., directly or indirectly through a care provider). The prescriptions and items can then be used for the home infusion therapy, to provide medical treatment to the patient. The home infusion therapy can be provided by the patient themselves, by a care provider to the patient (e.g., a nurse or physician), or in any other suitable manner.

FIG. 4 is a flowchart illustrating adding supplies for automated home infusion fulfillment, according to one embodiment. In an embodiment, FIG. 4 corresponds with block 310 illustrated in FIG. 3. At block 402, a fulfillment service (e.g., the fulfillment service 152 illustrated in FIGS. 1-2) identifies therapies relating to prescriptions. For example, the fulfillment service can receive information describing the therapies (e.g., a listing of therapies associated with a given patient), can retrieve the therapies from a repository (e.g., a repository describing therapies associated with a given patient), or can identify the therapies using any other suitable technique.

At block 404, the fulfillment service identifies supply options. In an embodiment, the therapies identified at block 402 are associated with supplies (e.g., in addition to prescriptions). For example, a repository (e.g., the therapies repository 140 illustrated in FIG. 1) can identify supplies associated with different therapies. In an embodiment, the fulfillment service identifies all supplies associated with the identified therapies. For example, the fulfillment service can use rules or other information to identify all supplies associated with each therapy, and can present all identified supplies to the user.

Alternatively, the fulfillment service identifies a subset of available supplies to present to the user. For example, the fulfillment service can identify recommended supplies. This recommendation can be based on patient information (e.g., patient medical history and demographic information), care provider information (e.g., care provider historical preferences, previous supply fulfilments, and other suitable information), pharmacist information (e.g., pharmacist preferences, historical pharmacist fulfillment information, and other suitable information), or any other suitable information.

As one example, supplies that have not been recently fulfilled for the patient or care provider (e.g., based on prior fulfillments) could be recommended. As another example, supplies frequently fulfilled for the given therapies (e.g., based on historical orders for the relevant parties, for others, or for both) could be recommended. As a third example, a suitable machine learning (ML) model could be trained to identify recommended supplies. For example, the ML model could use historical order information for any, or all, of the patient, care provider, treatment location, or pharmacist to identify recommended supplies. The ML model could be trained to infer recommended supplies, given a suitable combination of historical information, demographic information, or other information about the relevant entities.

Further, the fulfillment service can present a subset of supplies based on a threshold or maximum number of supplies suitable for the UI. In an embodiment, a UI can present a given number of supplies in a way that is accessible and visible to the user. The fulfillment service can identify that a given therapy is associated with more supplies than can be comfortably presented on the UI, and can present a subset of supplies. This subset can include recommended supplies (e.g., as discussed above), a pre-determined preference for supplies (e.g., supplies can be identified as higher or lower priority for presentation to users), a cropped listing of supplies (e.g., an initial number of supplies based on alphabetic listing or any other suitable organization), or any other suitable subset of supplies.

At block 406, the fulfillment service generates a UI to present the supplies for fulfillment. In an embodiment, FIG. 10 illustrates an example UI to add supplies. For example, the UI can present a list of relevant therapies for a given patient, and can present supplies related to each therapy.

At block 408, the fulfillment service fulfills the selected supplies. In an embodiment, a pharmacist is presented with a listing of supplies for selection (e.g., as illustrated in FIG. 10). The pharmacist can select which supplies should be fulfilled. In an embodiment, a subset of available supplies can be selected by default. This can be based on recommendations (e.g., using an ML model or any other suitable technique, as discussed above), pre-determined preferences, or any other suitable technique.

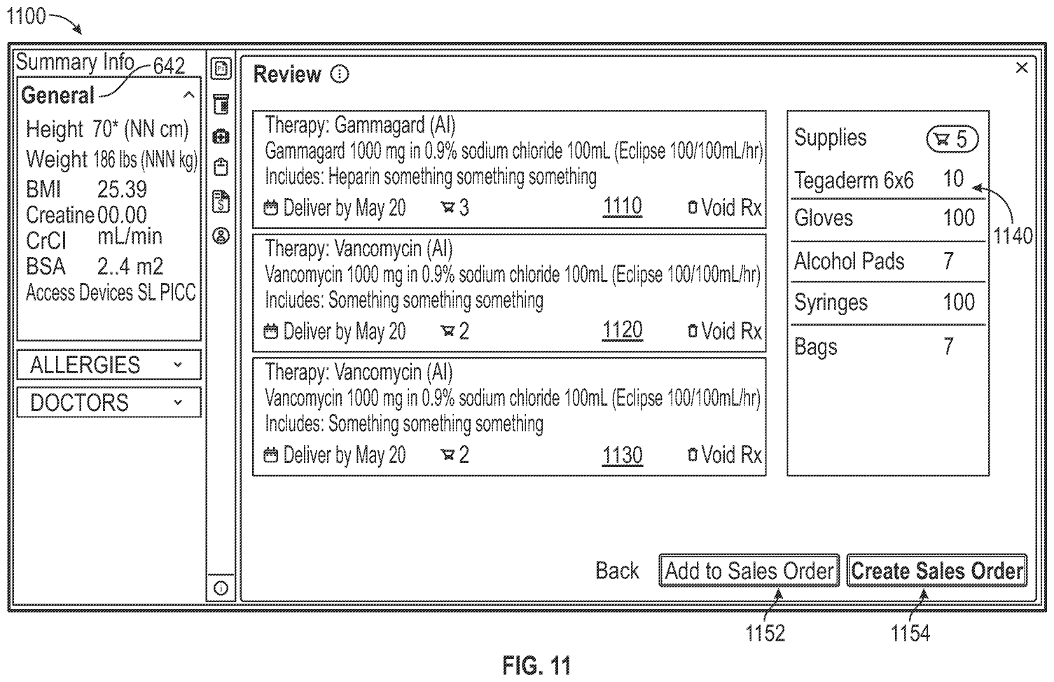

FIG. 5 is a flowchart illustrating generating orders for automated home infusion fulfillment, according to one embodiment. In an embodiment, FIG. 5 corresponds with block 312 illustrated in FIG. 3. At block 502, a fulfillment service (e.g., the fulfillment service 152 illustrated in FIGS. 1-2) presents the fulfillments for review. In an embodiment, the fulfillment service generates a UI listing the fulfillments that have been selected by the pharmacist. This includes both prescriptions and supplies. For example, FIG. 11 illustrates an example UI listing selected fulfillments for review.

At block 504, the fulfillment service determines whether to add the fulfillments to an existing order. In an embodiment, the pharmacist selects whether to add the fulfillments to an existing order. For example, FIG. 11 includes a button allowing the pharmacist to select whether to add the fulfillments to an existing order. Alternatively, or in addition, the fulfilment service can identify whether a fulfilled prescription exists, and can add the fulfillments to a pending order with the fulfilled prescription. For example, existing order can be maintained in a suitable repository and the fulfillment service can identify an existing order (e.g., for the relevant patient) in the repository.

If the fulfillment service determines to add fulfillments to an existing order, the flow proceeds to block 506. At block 506, the fulfillment service modifies the existing order. In an embodiment, the fulfillment service adds the selected prescriptions and supplies to the existing order.

Returning to block 504, if the fulfillment service determines that no existing order exists, the flow proceeds to block 508. At block 508, the fulfillment service creates a new order and adds the selected items to the order (e.g., prescriptions and supplies).

At block 510, the fulfillment service determines whether additional prescriptions are available (e.g., that have been previously filled). For example, a pharmacist may have previously filled prescriptions for a patient, but these previously filled prescriptions may not yet be attached to a sales order. The fulfillment service can present a UI and allow the pharmacist to add these filled prescriptions to the pending order

If the fulfillment service determines that additional prescriptions should be added (e.g., the pharmacist uses the UI to choose to add additional filled prescriptions), the flow proceeds to block 512. At block 512, the fulfillment service fulfills the remaining prescriptions. In an embodiment, the fulfillment service can add the remaining prescriptions to the pending sales order.

FIG. 6 illustrates a selection interface 600 for automated home infusion fulfillment, according to one embodiment. In an embodiment, the interface 600 is a UI used by a medical professional (e.g., a pharmacist) to select prescriptions for fulfillment. The interface 600 includes a list of prescriptions 610, including a first prescription 612, a second prescription 614, and a third prescription 616. This is merely an example, and any suitable number of prescriptions can be used. In an embodiment, the list of prescriptions 610 includes information relating to each of the prescriptions 612, 614, and 616, including an RX #(e.g., a prescription identifier), a number of doses remaining, a date last dispensed, a date for next fill or compound, an expiration date, and a status. In an embodiment, the list of prescriptions 610 can include any suitable information.

A pharmacist can select an action to use with prescription 612, 614, and 616 using a menu 630. For example, the pharmacist can copy, discontinue, hold, refill, or transfer a prescription. These are merely examples.

In an embodiment, three check boxes 620A-C allow the pharmacist to select which prescriptions are used with the selected action. For example, a pharmacist can select a refill action, and can select which of three prescriptions 612, 614, and 616 should be refilled using the check boxes 620A-C.

The interface 600 further includes a summary menu 640. The summary menu includes general information 642, allergy information 644, and doctor information 646. These are, again, merely examples and the summary menu 640 can present any suitable information.

FIG. 7 illustrates a prescription refill interface 700 for automated home infusion fulfillment, according to one embodiment. In an embodiment, the refill interface 700 is shown after a medical professional (e.g., a pharmacist) selects a prescription for refill fulfillment. In this example, a prescription 710 is one of three prescriptions selected for fulfillment.

The refill interface 700 includes a details menu 720, which provided details for the prescription. These details menu 720 include description information 722 (e.g., drug name, drug dose, container fluid, container volume, total volume, route, rate, infusion time, and pump information). These are merely examples, and the description information 722 can include any suitable information about the prescription.

In an embodiment, the details menu 720 further includes dosing information 724 (e.g., Sig information, starting date, frequency, duration, end date, and doses remaining information), and a variety of other information 726 (e.g., label instructions, prescriber information, origin information, and pharmacy notes). These are merely examples, and any suitable information can be used.

The interface 700 further includes a delivery information drop-down 730, compound directions drop-down 740, and insurance information drop-down 750. In an embodiment, each of these drop-downs can reflect missing, completed, required, or other status characteristics of the information. Further, the interface 700 includes an input 760 to save the prescription detail information as a template (e.g., a check box input) and a next button 770. In an embodiment, a pharmacist selects the next button 770 to move to the next UI screen.

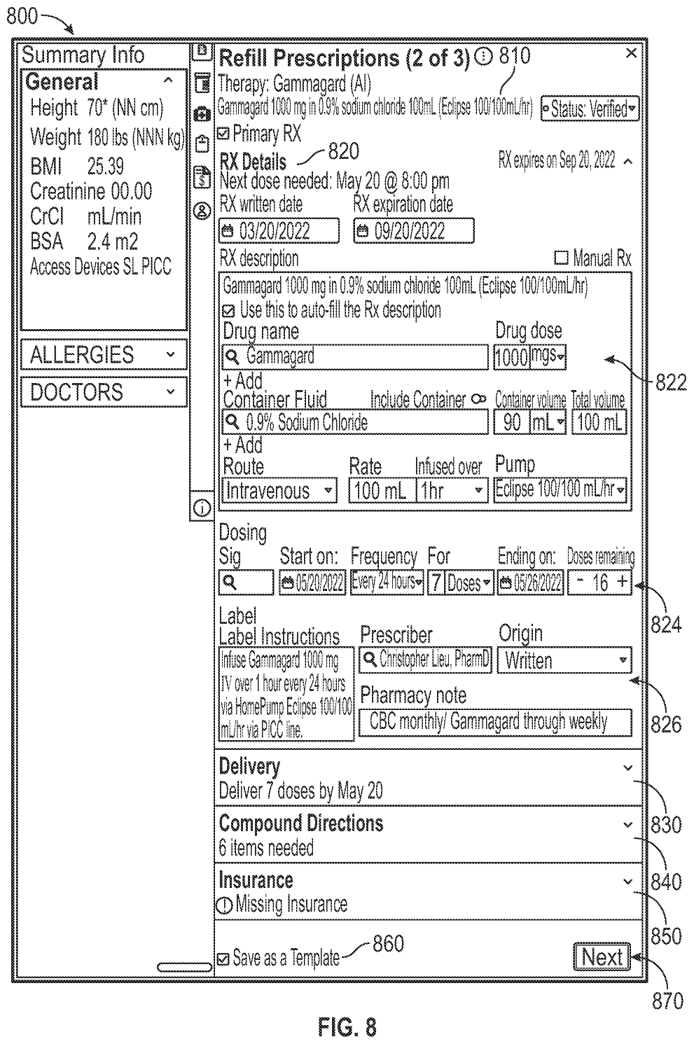

FIG. 8 illustrates a further prescription refill interface 800 for automated home infusion fulfillment, according to one embodiment. In an embodiment, the refill interface 800 reflects a next prescription 810 selected by a pharmacist for refill fulfillment. For example, the interface 800 can be presented to a pharmacist after selecting the next button 770 in the interface 700 illustrated in FIG. 7.

Like the interface 700 illustrated in FIG. 7, the refill interface 800 includes a details menu 820, description information 822, dosing information 824, and a variety of other information 826. These are merely examples, and any suitable information can be used.

The interface 800 further includes a delivery information drop-down 830, compound directions drop-down 840, and insurance information drop-down 850. In an embodiment, each of these drop-downs can reflect missing, completed, required, or other status characteristics of the information. Further, the interface 800 includes an input 860 to save the prescription detail information as a template (e.g., a check box input) and a next button 870. In an embodiment, a pharmacist selects the next button 870 to move to the next UI screen.

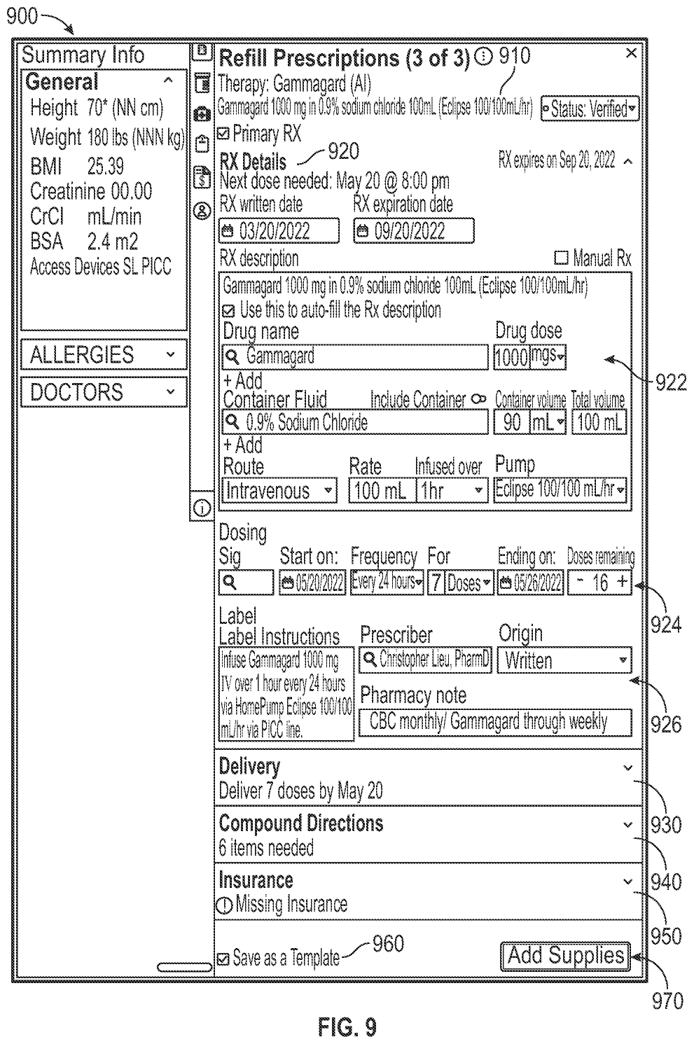

FIG. 9 illustrates another further prescription refill interface 900 for automated home infusion fulfillment, according to one embodiment. In an embodiment, the refill interface 900 reflects a next prescription 910 selected by a pharmacist for refill fulfillment. For example, the interface 900 can be presented to a pharmacist after selecting the next button 870 in the interface 800 illustrated in FIG. 8.

Like the interfaces 700 illustrated in FIGS. 7 and 800 illustrated in FIG. 8, the refill interface 900 includes a details menu 920, description information 922, dosing information 924, and a variety of other information 926. These are merely examples, and any suitable information can be used.

The interface 900 further includes a delivery information drop-down 930, compound directions drop-down 940, and insurance information drop-down 950. In an embodiment, each of these drop-downs can reflect missing, completed, required, or other status characteristics of the information. Further, the interface 900 includes an input 960 to save the prescription detail information as a template (e.g., a check box input) and a next button 970. In an embodiment, a pharmacist selects the next button 970 to move to the next UI screen.

FIG. 10 illustrates a supply fulfillment interface 1000 for automated home infusion fulfillment, according to one embodiment. In an embodiment, the interface 1000 allows a medical professional (e.g., a pharmacist) to add supplies associated with prescriptions being fulfilled. This is discussed further, above, with regard to block 310 illustrated in FIG. 3 and FIG. 4.

The interface 1000 includes three selection menus 1010, 1020, and 1030. Each of these selection menus lists an associated prescription and therapy, and allows a pharmacist to add items for fulfillment. For example, the selection menu 1030 includes items 1040A-N, which the pharmacist can choose to add for fulfillment. In an embodiment, the pharmacist can further select a quantity of each item for fulfillment. The interface 1000 further includes a supply menu 1050, which lists supplies that have been added for fulfillment.

In an embodiment, the interface 1000 can further include a status indication 1060 indicating whether a particular supply (or collection of supplies) is unavailable. For example, a fulfillment service (e.g., the fulfillment service 152 illustrated in FIGS. 1-2) can interact with a supply logistics system to identify which items are available and which items are unavailable. If a given item is unavailable, the fulfillment service can generate the status indication 1060. Further, the fulfillment service can indicate potentially similar items. The fulfillment service can identify similar items based on order history information, pre-defined relationships, rules, a suitable ML model (e.g., a trained ML model), or using any other suitable technique.

FIG. 11 illustrates a review interface 1100 for automated home infusion fulfillment, according to one embodiment. In an embodiment, the review interface 1100 includes three fulfillment menus 1110, 1120, and 1130. Each of these fulfillment menus 1110, 1120, and 1130 describes the prescription being fulfilled, the associated therapy, a delivery estimate, quantity information, and any other suitable information.

The review interface 1100 further includes a supplies menu 1140. In an embodiment, the supplies menu 1140 describes supplies selected for fulfillment (e.g., using the interface 1000 illustrated in FIG. 10).

The review interface 1100 further includes an add to sales order button 1152 and a create a sales order button 1154. As described above in relation to block 312 illustrated in FIG. 3, and FIG. 5, in an embodiment a fulfillment service (e.g., the fulfillment service 152 illustrated in FIGS. 1-2) can add the selected fulfillments to an existing order or create a new order. In an embodiment, the buttons 1152 and 1154 allow a pharmacist to select between these options.

If the pharmacist chooses to add the fulfillment to an existing order, the fulfillment service can generate a suitable UI to allow the pharmacist to select the existing order. For example, the fulfillment service can query a sales or logistics system to identify pending orders (e.g., for a particular patient). The fulfillment service can then present a list of available orders for the pharmacist to select from. Further, the UI can allow a pharmacist to search for an order (e.g., by order identifier, patient identifier, or other suitable search terms).

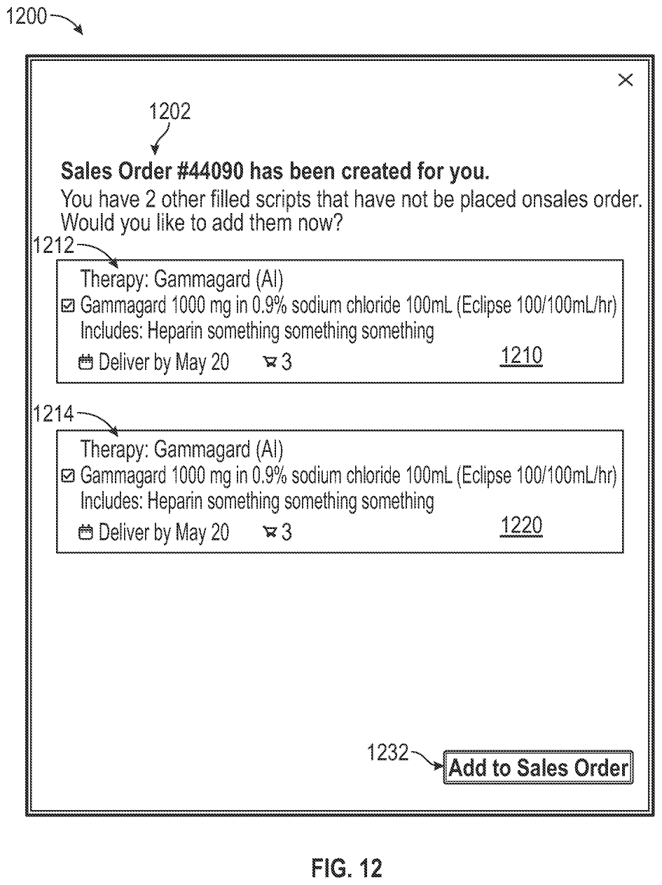

FIG. 12 illustrates an order interface 1200 for automated home infusion fulfillment, according to one embodiment. In an embodiment, the order interface 1200 presents an order identifier 1202 to identify the order. Further, as discussed above in relation to blocks 510 and 512 illustrated in FIG. 5, the order interface 1200 includes additional prescription menus 1210 and 1220 that can be added for fulfillment.

For example, a fulfillment service (e.g., the fulfillment service 152 illustrated in FIGS. 1-2) can identify prescription applicable to a given therapy or collection of therapies. If a pharmacist does not include some of these prescriptions for fulfillment, the interface 1200 can present these additional prescriptions using the additional prescription menus 1210 and 1220. For example, the pharmacist can select one or more of the check boxes 1212 and 1214 to choose the additional prescriptions for fulfillment. The pharmacist can then select a button 1232 to add the additional prescriptions to the sales order.

### Additional Considerations

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in FIGS., those operations may have corresponding counterpart means-plus-function components with similar numbering.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A computer-implemented method, comprising:
identifying a plurality of prescriptions for fulfillment by a pharmacist for a patient;
providing the plurality of prescriptions for presentation on a first screen of a user interface (UI);
receiving a selection of at least two of the plurality of prescriptions, based on input to the UI;
identifying one or more supplies associated with one or more of the at least two prescriptions, and providing the supplies for presentation on a second screen of the UI, wherein the second screen of the UI is accessible based on input to the first screen;
receiving a selection of a first supply of the one or more supplies, based on input to the UI; and
fulfilling the at least two prescriptions and the first supply, comprising:
triggering delivery of the at least two prescriptions and the first supply for treatment of the patient using a medical therapy.

2. The computer-implemented method of claim 1,
wherein the plurality of prescriptions and the one or more supplies all relate to a home infusion therapy, and
wherein the medical therapy comprises the home infusion therapy.

3. The computer-implemented method of claim 2, wherein the triggering delivery of the at least two prescriptions and the first supply comprises at least one of: (i) triggering delivery to the patient, or (ii) triggering delivery to a care provider associated with the patient, wherein the medical therapy is provided by the care provider.

4. The computer-implemented method of claim 2, wherein the identifying the plurality of prescriptions for fulfillment by the pharmacist for the patient comprises:
identifying, using an electronic repository, the plurality of prescriptions as relevant to the home infusion therapy.

5. The computer-implemented method of claim 2, wherein identifying one or more supplies associated with one or more of the at least two prescriptions comprises:
identifying, using an electronic repository, the one or more supplies as relevant to the home infusion therapy.

6. The computer-implemented method of claim 5, wherein the identifying one or more supplies associated with one or more of the at least two prescriptions further comprises:
identifying a plurality of supplies as relevant to the home infusion therapy; and
selecting the one or more supplies, from the plurality of supplies, based on historical information relating supply fulfillment.

7. The computer-implemented method of claim 1, wherein fulfilling the at least two prescriptions and the first supply further comprises:
receiving a selection, from the UI, to at least one of: (i) modify an existing sales order by adding the at least two prescriptions and the first supply or (ii) create a new sales order including the at least two prescriptions and the first supply; and
based on the selection, at least one of: (i) modifying the existing sales order by adding the at least two prescriptions and the first supply or (ii) creating the new sales order including the at least two prescriptions and the first supply.

8. An apparatus comprising:
a memory; and
a hardware processor communicatively coupled to the memory, the hardware processor configured to perform operations comprising:
identifying a plurality of prescriptions for fulfillment by a pharmacist for a patient;
providing the plurality of prescriptions for presentation on a first screen of a user interface (UI);
receiving a selection of at least two of the plurality of prescriptions, based on input to the UI;
identifying one or more supplies associated with one or more of the at least two prescriptions, and providing the supplies for presentation on a second screen of the UI, wherein the second screen of the UI is accessible based on input to the first screen;
receiving a selection of a first supply of the one or more supplies, based on input to the UI; and
fulfilling the at least two prescriptions and the first supply, comprising:
triggering delivery of the at least two prescriptions and the first supply for treatment of the patient using a medical therapy.

9. The apparatus of claim 8,
wherein the plurality of prescriptions and the one or more supplies all relate to a home infusion therapy, and
wherein the medical therapy comprises the home infusion therapy.

10. The apparatus of claim 9, wherein the triggering delivery of the at least two prescriptions and the first supply comprises at least one of: (i) triggering delivery to the patient, or (ii) triggering delivery to a care provider associated with the patient, wherein the medical therapy is provided by the care provider.

11. The apparatus of claim 9, wherein the identifying the plurality of prescriptions for fulfillment by the pharmacist for the patient comprises:
identifying, using an electronic repository, the plurality of prescriptions as relevant to the home infusion therapy.

12. The apparatus of claim 9, wherein identifying one or more supplies associated with one or more of the at least two prescriptions comprises:
identifying, using an electronic repository, the one or more supplies as relevant to the home infusion therapy.

13. The apparatus of claim 12, wherein the identifying one or more supplies associated with one or more of the at least two prescriptions further comprises:
identifying a plurality of supplies as relevant to the home infusion therapy; and
selecting the one or more supplies, from the plurality of supplies, based on historical information relating supply fulfillment.

14. The apparatus of claim 8, wherein fulfilling the at least two prescriptions and the first supply further comprises:
receiving a selection, from the UI, to at least one of: (i) modify an existing sales order by adding the at least two prescriptions and the first supply or (ii) create a new sales order including the at least two prescriptions and the first supply; and
based on the selection, at least one of: (i) modifying the existing sales order by adding the at least two prescriptions and the first supply or (ii) creating the new sales order including the at least two prescriptions and the first supply.

15. A non-transitory computer-readable medium comprising instructions that, when executed by a processor, cause the processor to perform operations comprising:
identifying a plurality of prescriptions for fulfillment by a pharmacist for a patient;
providing the plurality of prescriptions for presentation on a first screen of a user interface (UI);
receiving a selection of at least two of the plurality of prescriptions, based on input to the UI;
identifying one or more supplies associated with one or more of the at least two prescriptions, and providing the supplies for presentation on a second screen of the UI, wherein the second screen of the UI is accessible based on input to the first screen;
receiving a selection of a first supply of the one or more supplies, based on input to the UI; and
fulfilling the at least two prescriptions and the first supply, comprising:
triggering delivery of the at least two prescriptions and the first supply for treatment of the patient using a medical therapy.

16. The non-transitory computer-readable medium of claim 15,
wherein the plurality of prescriptions and the one or more supplies all relate to a home infusion therapy, and
wherein the medical therapy comprises the home infusion therapy.

17. The non-transitory computer-readable medium of claim 16, wherein the triggering delivery of the at least two prescriptions and the first supply comprises at least one of: (i) triggering delivery to the patient, or (ii) triggering delivery to a care provider associated with the patient, wherein the medical therapy is provided by the care provider.

18. The non-transitory computer-readable medium of claim 16, wherein the identifying the plurality of prescriptions for fulfillment by the pharmacist for the patient comprises:
identifying, using an electronic repository, the plurality of prescriptions as relevant to the home infusion therapy.

19. The non-transitory computer-readable medium of claim 16, wherein identifying one or more supplies associated with one or more of the at least two prescriptions comprises:
identifying, using an electronic repository, the one or more supplies as relevant to the home infusion therapy.

20. The non-transitory computer-readable medium of claim 15, wherein the second screen of the UI is accessible based on input to the first screen using one or more next buttons in the UI.
